(19) United States
(12) Patent Application Publication
(10) Pub. No.: US 2026/0263710 A1
(43) Pub. Date: Sep. 10, 2026

(54) MODIFIED VALVE FOR USE IN A FLOW PATH

(71) Applicant: Bayer Aktiengesellschaft, Leverkusen (DE)

(72) Inventors: Jared NEFF, Lower Burrell, PA (US); Gero EICHELKRAUT, Kreischa (DE)

(21) Appl. No.: 19/164,766

(22) PCT Filed: Mar. 8, 2024
(86) PCT No.: PCT/EP2024/056134

§ 371 Date: Sep. 12, 2025

Related U.S. Application Data
(60) Provisional application No. 63/490,363, filed on Mar. 15, 2023.

(51) Int. Cl. A61M 15/00; F16K 15/14

(52) U.S. Cl. CPC A61M 15/0015; F16K 15/148

(57) ABSTRACT

The invention relates to a modified umbrella valve and to a system for controlling flow in a flow path comprising the modified umbrella valve. The modified umbrella valve has a solid shaft, a first end and a second end. At the first end of the shaft is situated a flexible umbrella. At the second end of the shaft is situated a stop element having an enlarged diameter compared to the diameter of the shaft.

The invention relates to a valve for a breath-controlled inhaler for pulmonary inhalation.

Inhalers, which enable the patient to inhale an aerosol, are required for numerous medical applications, for example for the inhalational treatment of asthma, cystic fibrosis (CF) and a range of other respiratory diseases. An aerosol is a dispersion of small solid particles or liquid droplets in a continuous gas phase. Typically, medical inhalational treatments require aerosols of fine droplets of a liquid formulation of a bioactive active ingredient or drug that ideally also reach the tiniest branches of the peripheral lungs, such as bronchioles and alveoli.

In order to achieve the desired homogeneous distribution of droplets in the gas phase, the liquid formulation is atomized in the inhaler by a nebulizer, for example ultrasonic nebulizer, nozzle nebulizer or membrane nebulizer.

Components of the nebulizers currently available are, for example, a reservoir for a liquid or for solid particles, an aerosol generator, a mixing chamber, a mouthpiece, and an air way between an air inlet and the mouthpiece. Depending on the design of the inhaler, there may be yet further components, for example for controlling the air flow.

Various inhalers are known from the prior art:
WO 2006/083014 A1; U.S. Pat. Nos. 11,534,559; 11,458,264 B2; US 2021/0252236, US 2022/0126036 A1; WO2013/132056.

With these breath-controlled inhalers, an air flow in the inhaler is generated by the patient upon inhalation at the mouthpiece. The air flow mixes with the aerosol generated by the aerosol generator, and the mixture of air and aerosol is transported to the mouthpiece via an air channel. With some inhalers, the patient is supposed to remove the mouthpiece of the inhaler upon exhalation and to reapply it for inhalation. However, the patients frequently exhale into the mouthpiece and hence into the air way in the inhaler. As a result, respiratory air can reach the interior of the inhaler, where it can cause damage in the long run through moisture or, in the case of powder inhalers, moisten or swirl the powder.

Therefore, many of the known inhalers have exhalation valves, which prevent the exhaled air from reaching, through the mouthpiece, the interior of the inhaler, in particular the reservoir for liquid or powder, but also the control electronics, and enable the users to exhale into the mouthpiece. The exhalation valves used are duckbill valves or flapper valves, for example in US2022/0126036 A1, US2021/0252236 A1, U.S. Pat. No. 11,458,264 B2. During inhalation, when an air flow is generated from the air inlet to the mouthpiece, these valves are open, and during exhalation, when an air flow is generated from the mouthpiece toward the interior of the inhaler, the exhalation valves close. Exhalation valves can also be used to divert the gas flow into the surroundings (environment) during exhalation.

U.S. Pat. No. 11,534,559 B2 discloses an umbrella valve as a breath-controlled air inlet valve that opens in the event of a negative pressure in the air inlet chamber of the inhaler.

Valves which are present in the air way influence the air flow during inhalation, since they are an obstacle in the air channel. Therefore, the valve should be designed in such a way that it affects the air flow as little as possible during inhalation and provides optimal blocking of the air way during exhalation.

The modified umbrella valve according to the invention particularly meets this requirement. Furthermore, the invention consists in a system for controlling flow in a flow path comprising a modified umbrella valve. As an example, a breath-controlled inhaler comprising a modified umbrella valve and the use of the modified umbrella valve in the air channel of an inhaler is described in the specification. It is understood that the modified umbrella valve and the system for controlling flow in a flow path are not necessarily tied to be used with air and in an air channel but could very well be used with other gases and generally in gas flow channels. Therefore “air channel” or “air way” or “air flow” shall also be understood in the broader meaning of “gas channel” or “gas way” or “gas flow”, as the case may be, unless specifically used with relation to the breath-controlled inhaler as described below.

The modified umbrella valve is a check valve. It comprises a solid shaft having a first end and a second end. At the first end of the shaft is situated a flexible umbrella. At the second end of the shaft is situated a stop element having an enlarged diameter compared to the diameter of the shaft.

During use, the modified umbrella valve is mounted in the central hole of a perforated plate, the perforated plate being situated in an air channel. The perforated plate can be of differing thickness, porosity and diameter. The diameter of the flexible umbrella substantially corresponds to the diameter of the air channel or also provides radial sealing thereof via a slight oversize. The diameter of the stop element should be greater than the diameter of the central hole of the perforated plate into which it is inserted. The stop element can be angled to allow for ease of insertion. This is the only way that the stop element holds the second end of the shaft on the upstream side of the perforated plate. At the same time, the stop element should not be so large that it affects the air flow through the air channel, for example it should cover as few perforations of the perforated plate as possible.

If an air flow in the air channel hits the outside of the umbrella of the umbrella valve (positive pressure), the umbrella is pressed onto the perforated plate and closes the perforations of the perforated plate in axial direction. In addition, an additional seal can be formed by a slight oversize of the outer diameter of the umbrella valve in relation to the inner diameter of the air channel. If, by contrast, a negative pressure is generated on the outside of the umbrella, the umbrella is lifted up from the perforated plate and folds down into the direction opposite to the perforated plate. At the same time, the effective diameter of the umbrella decreases and a portion of the air channel diameter is released for an air flow through the perforated plate. The first end of the shaft moves away from the perforated plate until further movement is stopped by the stop element, in accordance with the length of the shaft.

The length of the shaft should be chosen such that the folded-down umbrella is as far away as possible from the perforated plate in order to reduce the air flow through the perforated plate as little as possible. The shaft and the stop feature are to hold the umbrella portion of the valve in position to the perforated plate. On the other hand, an excessively long distance of the folded-down umbrella from the perforated plate involves the risk of the umbrella tilting when the negative pressure decreases and not correctly returning to the seat on the perforated plate. Preferably, the length of the shaft should therefore lie in the range from 20% to 50%, particularly preferably in the range from 30% to 40%, of the diameter of the umbrella. This allows for linear movement of the umbrella value to improve air flow during inhalation. On larger diameters, this ratio of shaft to diameter of the umbrella can be further increased or reduced based on the application and the inhalation and exhalation forces the umbrella valve is exposed to.

In one embodiment in which the diameter of the umbrella lies in the range from 9.5 mm to 10.5 mm, the shaft is at least 2 mm, preferably at least 2.5 mm, particularly preferably at least 3 mm long. In said embodiment, the shaft is at most 5 mm, preferably at most 4 mm long. Particularly preferably, the shaft in said embodiment is in the range from 3 mm to 4 mm long.

The umbrella of the umbrella valve is preferably composed of silicone or a similarly flexible material having sealing properties such as thermoplastic elastomer (TPE) or thermoplastic urethane (TPU). The shaft can consist of the same material or a rigid plastic such as PEEK (polyetheretherketone) or PC (polycarbonate) or of metal. The stop element can be formed integrally with the shaft, composed of the same material as the umbrella, or it can be attached to the second end of the shaft and composed of a different material. The stop element is one which prevents the second end of the shaft from sliding through the hole of a perforated plate. The umbrella can likewise be formed integrally with the shaft, for example by manufacture by multicomponent injection molding with a form-fit connection or material-to-material bonding, or as a separate component which, for example, is attached to the shaft after the shaft has been fitted in the guide of the perforated plate. The number of individual parts of which the modified umbrella valve consists depends on how it is to be fitted.

A system for controlling flow in a flow path according to the invention comprises
an air channel;
a perforated plate situated in the air channel and having a central hole;
a modified umbrella valve as described above mounted in the central hole of the perforated plate.

In one embodiment the diameter of the flexible umbrella of the modified umbrella valve corresponds to the diameter of the air channel.

In one embodiment the diameter of the stop element is greater than the diameter of the central hole of the perforated plate into which it is inserted.

In one embodiment the length of the shaft is between 1.2 to 5 times the thickness of the perforated plate, preferably between 2 to 4 times the thickness of the perforated plate, most preferred between 2 to 3 times the thickness of the perforated plate.

The exemplary breath-controlled inhaler comprises
an air inlet,
a mouthpiece having a mouthpiece opening,
an air channel between air inlet and mouthpiece opening, wherein the air channel comprises a mixing chamber,
a reservoir for a liquid or for solid particles,
an aerosol generator which is connected to the reservoir and converts the liquid or the solid particles from the reservoir into an aerosol and introduces it into the mixing chamber,
a modified umbrella valve between air inlet and mixing chamber.

In one embodiment, the modified umbrella valve is mounted in the central hole of a perforated plate. The perforated plate is situated in the air channel between air inlet and mixing chamber. The perforated plate can, at the same time, also form the function of an air filter for retention of particles or moisture and comprise an appropriate mesh. The plate has typically a thickness between 0.3 mm and 3 mm.

The breath-controlled inhaler preferably comprises an air inlet valve between the modified umbrella valve and the air inlet. The air inlet valve is controlled via a pressure sensor which releases the air inlet in the event of a certain negative pressure in the mouthpiece.

The aerosol generator is preferably an electromechanical aerosol generator comprising a vibrating membrane which seals off the reservoir toward the mixing chamber. As a result of vibration, the aerosol is generated (membrane nebulizer), which mixes with the airflow in the air channel.

In one embodiment, the breath-controlled inhaler according to the invention is made up of a head part, a base part and a mouthpiece. The head part comprises the reservoir and the aerosol generator, and the base part comprises the air inlet and a first section of the air channel. Furthermore, the base part can comprise the air inlet valve, electronics for operation of the aerosol generator, and a pressure sensor. The mouthpiece comprises the mouthpiece opening, the second section of the air channel with mixing chamber, and the modified umbrella valve at the start of the 2nd section of the air channel.

The position of the modified umbrella valve at the entrance of the 2nd section of the air channel prevents possible penetration of exhaled air from the mouthpiece opening right into the 1st section of the air channel in the base part and prevents electronics present there from being damaged by moisture. This can occur through accidental exhalation into the device by the user. Even if gaskets are present, there may be leaks. This is because of the complexity of the sealing interfaces between components and because of the tolerance difference between the components. This prevents a proper alignment and application of force to elastomer gaskets, which is necessary to ensure leak-tightness. The moisture can also originate from the surroundings, for example if the inhaler is being used in a warm room with high air humidity, after it has been transported at cold temperatures. Damage to the electronics in the base part is particularly critical because the base part is usable multiple times and is intended to remain fit for use for a relatively long period, whereas the mouthpiece and the head part are exchanged regularly. In addition, the position of the modified umbrella valve between air inlet and mixing chamber prevents the aerosol-laden airflow on the way to the mouthpiece opening from being affected.

At the mouthpiece, the patient generates the airflow, into which the aerosol generated by the vibrating membrane is admitted. Via the mouthpiece opening, the mixture of aerosol and air is inhaled by the patient. The necessary air is sucked in via an air inlet opening into the air channel in the base part, from where it reaches the air channel of the mouthpiece.

### FIGURES AND EXAMPLES

Exemplary embodiments of the invention are depicted in the figures and will be more particularly elucidated below. In the figures:

FIG. 1a shows an umbrella valve from the prior art in a closed position

FIG. 1b shows an umbrella valve from the prior art in an open position

FIG. 2 shows umbrella valves having a short shaft and having a long shaft

FIG. 3a shows a modified umbrella valve in the idle state in the first variant of an air channel of an inhaler

FIG. 3b shows a modified umbrella valve during inhalation in the first variant of an air channel of an inhaler

FIG. 3c shows a modified umbrella valve during exhalation in the first variant of an air channel of an inhaler

FIG. 4a shows a modified umbrella valve in the idle state in the second variant of an air channel of an inhaler

FIG. 4b shows a modified umbrella valve during inhalation in the second variant of an air channel of an inhaler

FIG. 4c shows a modified umbrella valve during exhalation in the second variant of an air channel of an inhaler

FIG. 5 shows an exploded drawing of an inhaler from the prior art

FIG. 6 shows a longitudinal section through an inhaler from the prior art

FIG. 7 shows measurement results relating to moisture in the inhaler from the prior art without an exhalation valve

FIG. 8 shows measurement results relating to moisture in the inhaler from the prior art with a modified umbrella valve

### REFERENCE SIGNS

10—Umbrella valve from the prior art
12—Shaft end, umbrella valve from the prior art
14—Shaft, umbrella valve from the prior art
15—Hook, umbrella valve from the prior art
16—Umbrella, umbrella valve from the prior art
18—Perforated plate
19—Perforation
20—Modified umbrella valve
23—Stop element
24—Shaft
26—Umbrella
28—Central hole
29—Perforated plate or filter plate
100—Base unit
101—Air inlet opening
102—Air outlet opening
103—Groove
104—Key locking element
110—Air channel
118—Air inlet valve
119—Pressure sensor
200—Mouthpiece
200a—First segment of the mouthpiece
200b—Second segment of the mouthpiece
201—Air intake opening
202—Lateral opening
203—Mouthpiece opening
206—Step
207—Air channel
208—Mixing chamber
300—Aerosol head
301—Aerosol generator
302—Reservoir
303—Key locking element
304—Lid
309—Mesh

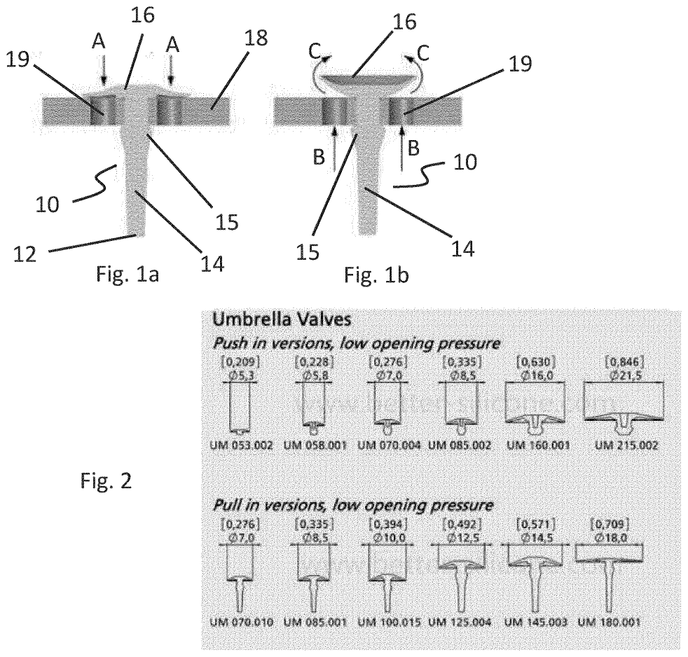

FIG. 1a shows an umbrella valve 10 from the prior art in a closed position. The shaft 14 sits in the central hole of a perforated plate 18, where it is fixed on one of the sides of the perforated plate 18 by the umbrella 16 and on the other side of the perforated plate 18 by the hook 15. The hook 15 is a widening of the shaft 14. The shaft end 12 is spaced from the umbrella 16, so that the shaft 14 of the umbrella valve 10 when fitted can be pulled through the central hole of the perforated plate 18. In the closed position, the umbrella 16 of the umbrella valve 10 closes the perforations 19 in the perforated plate 18. The umbrella valve 10 is in the closed position when a medium (gas or liquid) exerts pressure on the outside of the umbrella 16 (arrow A). From a certain pressure of a medium into the opposite direction through the perforations 19 of the perforated plate 18 onto the bottom side of the umbrella 16 (arrow B), the umbrella 16 folds down. The outer regions of the umbrella 16 lift up from the perforations 19, so that the medium (air or liquid) can flow through the perforations 19 (FIG. 1b, arrow C).

FIG. 2 shows not only umbrella valves having a long shaft, but also umbrella valves having a short shaft. When fitted, they are not pulled through the central hole of a perforated plate but are one-sidedly pressed in until the hook engages. The shaft serves as a fitting aid and as a support by means of an undercut. It has little axial movability purely for fitting purposes. In the prior art, it does not perform the function of axial slidability under negative/positive pressure in the air channel for better clearance of the flow cross section.

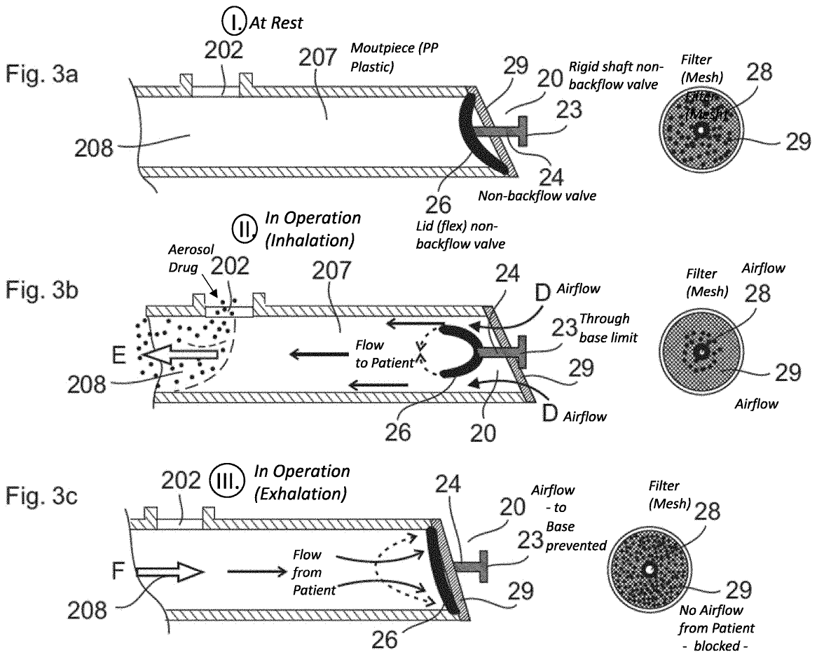

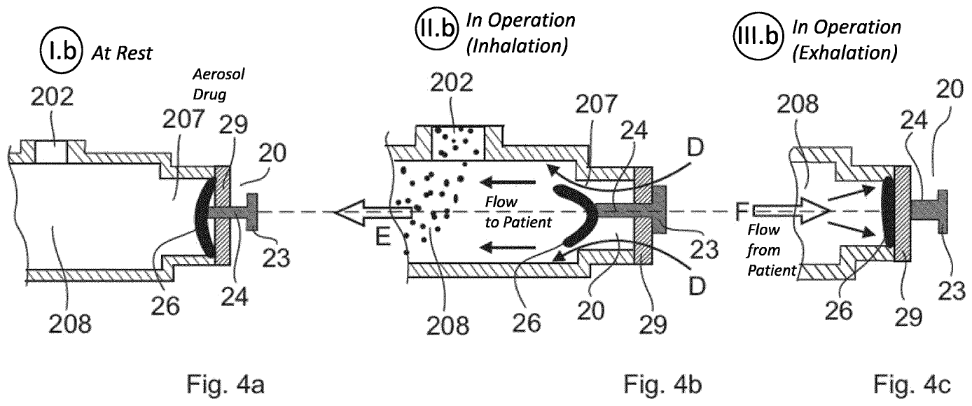

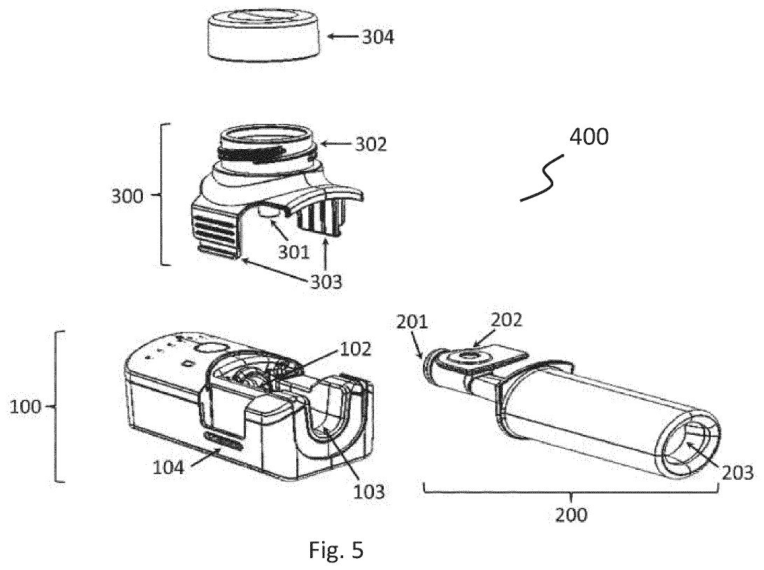

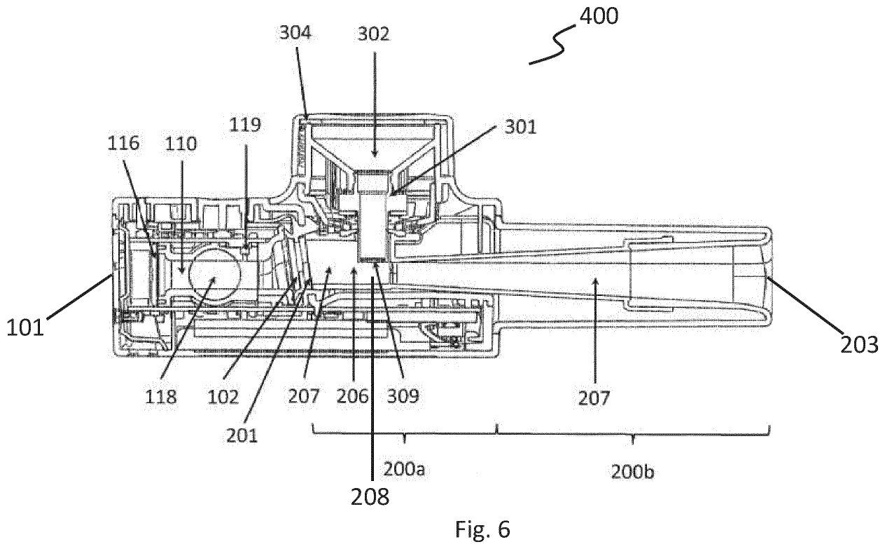

FIGS. 3a and 4a show the modified umbrella valve 20 in the idle state as part of a system for controlling flow in a flow path, which could for example be present in the inhaler 400 (FIGS. 5 and 6). The two variants of the air channels 207 in FIGS. 3a-c and 4a-c differ in that the air channel 207 in FIGS. 3a-c has a constant diameter, whereas the air channel in FIGS. 4a-c has a step, so that the air channel widens between perforated plate or filter plate 29 and mixing chamber 208.

The modified umbrella valve 20 comprises a shaft 24 and, at the first end thereof, an umbrella 26. The modified umbrella valve 20 differs from a known umbrella valve 10 in that a stop element 23 is situated at the second end of the shaft 24. Furthermore, there is no hook 15 present as in the case of the umbrella valves 10 from the prior art, which hook fixes the shaft 24 to the perforated plate or filter plate 29 in a fixed position. In the idle state, the umbrella 26, as a consequence of its natural tension, is arched toward the shaft 24 and is situated in an undefined position in the air channel 207 at a maximum distance from the perforated plate or filter plate 29 that is limited by the stop element 23. In FIG. 3a and FIG. 4a, the umbrella of the modified umbrella valve in the idle state is situated in a position directly on the perforated plate or filter plate 29. The shaft 24 is mounted in the central hole 28 of a perforated plate or filter plate.

FIGS. 3b and 4b show the situation when an airflow is generated in the direction of arrow D through the perforated plate or filter plate 29. The air flows through the perforated plate or filter plate 29 and, in doing so, lifts up the umbrella 26 of the umbrella valve 20. Owing to the air flow, the umbrella 26 is removed from the perforated plate or filter plate 29 as far as is allowed by the stop element at the second end of the shaft 24. Thereafter, the umbrella 26 of the umbrella valve 20 folds down away from the shaft 24 in the direction of the airflow D. Now, the air flows relatively unimpeded further down the flow path for example into the mixing chamber 208, in which the air flow mixes with the aerosol from the lateral opening 202 connected to the aerosol generator 301. The air-aerosol mixture then flows in the further direction of the mouthpiece opening 203 of the inhaler (arrow E).

If an air flow is generated in the opposite direction (FIG. 3c/FIG. 4c, arrow F), the umbrella 26 is pressed onto the perforated plate or filter plate 29. At the same time, the umbrella 26 closes the perforations in the perforated plate or filter plate 29 in an axial direction and the air flow comes to a standstill. An additional sealing effect can be produced radially by a slight oversize of the umbrella valve in relation to the inner diameter of the air channel.

FIG. 5 shows an exploded drawing and FIG. 6 shows a longitudinal section of an inhaler 400 from the prior art (EP 2724741 B1 ) that has been improved by fitting the modified umbrella valve 20 at a suitable position. The inhaler 400 comprises a base unit 100, a mouthpiece 200 and an aerosol head 300.

The base unit 100 comprises one or more air inlet openings 101, an air outlet opening 102, a groove 103 for accommodation of the mouthpiece 200, and one or more key locking elements 104. The air inlet openings 101 and the air outlet opening 102 are connected inside the base unit 100 to form an air channel 110. Under operating conditions and during the inspiration phase of a user, air flows from the upstream end toward the downstream end of the air channel 110, i.e. from the air inlet openings 101 to the air outlet opening 102.

A pressure sensor 119 measures the negative pressure of inhalation and activates the aerosol generator 301 from a certain minimum pressure and opens/closes the air inlet valve 118 in order to control the air flow. The pressure sensor 119 is in contact with the air way as an orthogonal bypass in relation to the main gas flow of the air flow in the air channel 110.

The mouthpiece 200 comprises a first segment 200a, comprising an air intake opening 201 attachable to the air outlet opening 102 of the base unit 100, and a lateral opening 202 for connection to the aerosol generator 301. Following the air intake opening 201 is an air channel 207, which expands in the region of the lateral opening to a mixing chamber 208. In the mixing chamber 208, during use, the air mixes with the aerosol mixture from the aerosol head 300. The first segment 200a is insertable into the groove 103 of the base unit 100. A second segment 200b is situated downstream of the first segment 200 a and comprises the continuation of the air channel 207 from the first segment 200a and the mouthpiece opening 203.

The aerosol head 300 comprises an aerosol generator 301, a reservoir for a liquid 302, and one or more male or female key locking elements 303 complementary to the key locking elements 104 on the base unit 100. The base unit 100, the mouthpiece 200 and the aerosol head 300 are interconnectable.

The assembly of the base unit 100 with mouthpiece 200 and aerosol head 300 is achieved by insertion of the first segment 200a of the mouthpiece 200 into the groove 103 of the base unit 100, followed by placement of the aerosol head 300 over the first segment 200a of the mouthpiece 200 and engagement of the key locking element(s) 303 of the aerosol head 300 with the complementary element(s) 104 of the base unit 100 through gentle pressure both on the aerosol head and on the base unit. These few assembly steps are intended to produce air-tight connections between the air outlet opening 102 of the base unit 100 and the air intake opening 201 of the first segment 200a of the mouthpiece and also between the aerosol generator 301 and the lateral opening 202 in the first segment 200a of the mouthpiece 200. Furthermore, the aerosol generator 301 is positioned in the aerosol head 300 such that the aerosol generator 301, upon engagement of the key locking element(s) with the complementary element(s), is at least partially inserted into the lateral opening 202 of the first segment 200a of the mouthpiece 200.

Through the outlet opening 102 of the base unit 100, air flows into the air channel 207 of the mouthpiece 200 via the air intake opening 201 of the first segment 200a of the mouthpiece 200. The air outlet opening 102 is circular and can, for example, be positioned in a central region of the base unit (as shown in FIG. 5 for example). The air outlet opening 102 can comprise a sealing element, for example in the form of a gasket, a sealing lip or bellows, which serves to establish an air-tight connection with the air intake opening 201 of the mouthpiece 200.

The air intake opening comprises a perforated plate or filter plate 29 having a central hole 28, which accommodates the shaft 24 of a modified umbrella valve 20. At the same time, the umbrella 26 is situated in the air channel 207 of the mouthpiece 200 and the stop element 23 is situated in the region of the air outlet opening 102 of the base unit 100 (see also FIG. 3a-3c/FIG. 4a-c). The modified umbrella valve 20 prevents especially air flow formed upon exhalation into the mouthpiece opening from passing through possibly incomplete seals, across the air intake opening 201 and the air outlet opening 102, into the base unit 100.

The base unit 100 can comprise one or more electrical connectors for electrical connection thereof, in the assembled state, with complementary connectors of the aerosol head 300. This is particularly important if the aerosol head 300 comprises an ultrasonic or piezoelectric aerosol generator 301, such as an aerosol generator 301 comprising a vibrating membrane 309 (membrane nebulizer).

The power supplied to the aerosol generator can, for example, be provided by an internal battery or a rechargeable battery housed in the base unit 100 or by an external energy source connected via a cable to the base unit 100.

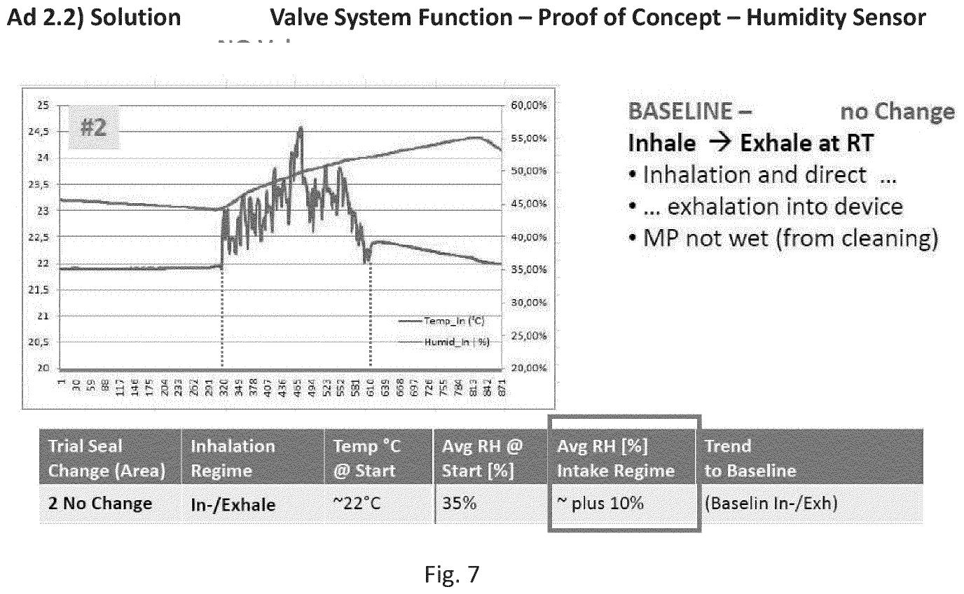

FIG. 7 shows measurement results relating to moisture in the inhaler from the prior art without the modified umbrella valve 20. Situated in the base part 100 was a moisture sensor near the pressure sensor 119. The user inhaled through the mouthpiece 200 and exhaled immediately afterwards. From the graph in FIG. 6, it can be seen that the moisture sensor measured an elevated relative air humidity in the exhalation phase (time unit 320 to 610 on the x-axis). From 35% rel. air humidity at 23° C., rel. air humidity rose in the peak up to 57%. On average, the increase in relative air humidity at the sensor during the period of exhalation was about 10%.

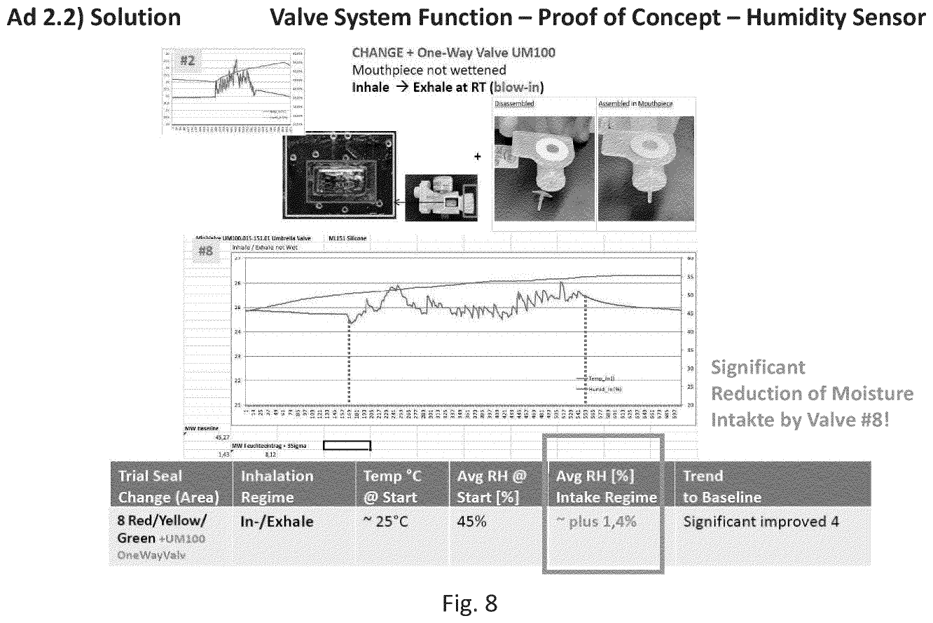

FIG. 8 shows measurement results relating to moisture in the inhaler from the prior art with a modified umbrella valve 20. Exactly as in the test without a modified umbrella valve, the user inhaled through the mouthpiece and exhaled immediately afterwards. From the graph in FIG. 7, it can be seen that the moisture sensor measured a slightly elevated relative air humidity in the exhalation phase (time unit 169 to 553 on the x-axis). The increase in relative air humidity was distinctly lower than without the modified umbrella valve 20. From 45% rel. air humidity at 25° C., rel. air humidity rose in the peak up to 54%. On average, the increase in relative air humidity at the sensor was about 1.4%.

1. A modified umbrella valve comprising a solid shaft having a first end and a second end, wherein at the first end of the shaft is situated a flexible umbrella, wherein at the second end of the shaft is situated a stop element having an enlarged diameter compared to the diameter of the shaft

2. The modified umbrella valve as claimed in claim 1, wherein the length of the shaft lies in the range from 20% to 50%, of the diameter of the umbrella.

3. The modified umbrella valve as claimed in claim 1, wherein the diameter of the umbrella lies in the range from 9.5 mm to 10.5 mm and the shaft is at least 2 mm long.

4. The modified umbrella valve as claimed in claim 1, wherein the diameter of the umbrella lies in the range from 9.5 mm to 10.5 mm and the shaft is at least in the range from 3 mm to 4 mm long.

5. The modified umbrella valve as claimed in claim 1, wherein the umbrella is composed of flexible materials selected from the group consisting of silicone, thermoplastic elastomers (TPE), and thermoplastic urethanes (TPU).

6. The modified umbrella valve as claimed in claim 1, wherein the shaft consists of a metal or of a hard plastic, wherein the hard plastic is PEEK (polyetheretherketone) or PC (polycarbonate).

7. The modified umbrella valve as claimed in claim 1, wherein the umbrella is formed integrally with the shaft.

8. A system for controlling flow in a flow path comprising:
a gas channel;
a perforated plate situated in the gas channel and having a central hole;
a modified umbrella valve mounted in the central hole of the perforated plate;
wherein the modified umbrella valve comprises a solid shaft having a first end and a second end, wherein at the first end of the shaft is situated a flexible umbrella, and at the second end of the shaft is situated a stop element having an enlarged diameter compared to the diameter of the shaft.

9. The system as claimed in claim 8, wherein the diameter of the flexible umbrella corresponds to the diameter of the gas channel.

10. The system as claimed in claim 8, wherein the diameter of the stop element is greater than the diameter of the central hole of the perforated plate into which it is inserted.

11. The system as claimed in claim 8, wherein the length of the shaft is between 1.2 to 5 times the thickness of the perforated plate.

12. The system as claimed in claim 11, wherein the length of the shaft is between 2 and 4 times the thickness of the perforated plate.

13. The system as claimed in claim 11, wherein the length of the shaft is between 2 and 3 times the thickness of the perforated plate.
